(19) United States
(12) Patent Application Publication
(10) Pub. No.: US 2014/0195258 A1
(43) Pub. Date: Jul. 10, 2014

(54) METHOD AND SYSTEM FOR MANAGING ENTERPRISE WORKFLOW AND INFORMATION

(71) Applicant: Wisdom Tools Enterprises, Inc., Bloomington, IN (US)

(72) Inventors: Matthew M. Burton, Bloomington, IN (US); Steven C. Borland, Bloomington, IN (US); Eugene H. Kirkley, JR., Bloomington, IN (US)

(21) Appl. No.: 14/040,342

(22) Filed: Sep. 27, 2013

Related U.S. Application Data
(63) Continuation of application No. 12/171,184, filed on Jul. 10, 2008, now abandoned.
(60) Provisional application No. 60/948,924, filed on Jul. 10, 2007.

(51) Int. Cl. G06Q 10/06; G06Q 50/22

(52) U.S. Cl. CPC G06Q 10/0633; G06Q 50/22
USPC 705/2; 705/7.26

(57) ABSTRACT

A system for enterprise workflow management includes software and hardware for gathering information regarding the current state of workflows within the enterprise, examining the operational relationships among the systems and entities relating to the workflows, and facilitating improvement of the workflows throughout their respective lifecycles.

### CROSS-REFERENCE TO RELATED APPLICATIONS

The present application is a continuation of U.S. patent application Ser. No. 12/171,184, filed Jul. 10, 2008, which claims priority to U.S. Provisional Patent Application Ser. No. 60/948,924, entitled “HOLISTIC SOLUTIONS SYSTEM,” filed Jul. 10, 2007, the entire contents of the disclosures of which are hereby expressly incorporated herein by reference.

### FIELD OF THE DISCLOSURE

The present disclosure generally relates to a systems life-cycle management based enterprise operations and information technology solution, and more particularly to a method and system for managing enterprise workflow and information by clearly defining existing workflows to permit detailed analysis, intervention, simulation, training, and optimization.

### BACKGROUND OF THE DISCLOSURE

Conventional health care delivery systems in hospitals, clinics, and centers are extremely complex environments that are typically managed without a system-wide and detailed understanding of their daily operations and the ever evolving processes, tools, and technologies supporting these activities. This lack of understanding often creates an overwhelming challenge for all levels of management in their efforts to improve quality, maintain patient safety, and function efficiently in this intricate and highly technical enterprise. Current technology providers design and deliver products with little attention to or knowledge of the actual clinical workflows involved in these daily operations. While some providers purport to automate “workflow,” they generally fail to first define or understand true clinical workflow—the progression and combination of physical, communicative, and cognitive tasks taken to achieve short, medium, and long term clinical and operational outcomes.

The above-mentioned poorly thought-out or even carelessly designed software applications cause numerous serious issues with the workflow of healthcare providers. Additionally, they are often inflexible and have very long adjustment cycles (often a decade or longer) as the delivery of care continues to change at an ever increasing pace. Furthermore, it has even been clearly demonstrated that careless implementation of such technology can result in very negative outcomes for patients including severe injury or even death, thus the emergence of a new cause of hospital acquired illness termed e-latrogenisis.

As an example of the complexity of these environments, on today's inpatient ward, it is not atypical for 8-16 patients to be directly cared for by one to two nurses with help from various ancillary staff and under the direction of five to ten different physicians from different specialties and with difference practice preferences and training. Each patient often has multiple co-morbid and/or unrelated diseases as well as numerous pharmaceutical and/or surgical interventions (past, present and planned) at various stages of severity, progression, and resolution. Their physiological and pathological state is continuously in flux, measured directly or indirectly (or even not at all) by a variety clinical tests (e.g., lab, radiology, monitors). Making matters worse, roughly every eight to twelve hours the individual nurses and personnel change. Moreover, these personnel are often trained weekly on new policies, procedures, best practices, and/or technologies. Multiply this by literally dozens of wards or departments, some performing very advanced and specialized interventions, and the result is a description of chaos. Now, introduce computer systems designed with insufficient consideration for domain specific knowledge and even less for local workflow with acceptable (or even necessary) variations that occupy as much of the clinicians time as the patient.

At best, conventional business analytic/intelligence tools, which focus on outcomes measurement, fail to provide the necessary tools for improving the very means (processes, people, policies, environment, etc.) by which these outcomes are achieved. This forces administrators and quality improvement personnel to use manual data collection and analysis methodologies that consume valuable human resources, are wrought with opportunity for error, and often deliver sub-optimal results or entirely missed opportunities. Directors of nursing have openly admitted that they know that nurse behavior changes when the nurse is being watched (Hawthorne effect) and that they have no way of analyzing workflow over time (Snapshot View). Furthermore, many conventional process improvement methodologies (e.g., LEAN) involve conducting the initiative in the “place of work,” such as a factory. Patients, however, certainly are not products, just as hospitals are not factories. Patients are, by definition, unique entities and have personal preferences. There are many issues with conducting such activities at the point of care (e.g., infection control), not the least of which is patient privacy or hospitality experience.

### SUMMARY OF THE DISCLOSURE

The present disclosure provides methods and systems for acquiring a system-wide, knowledge based, detailed understanding of enterprise workflows, and incorporating various management, training and simulation tools for analyzing and optimizing the workflows to improve inefficiencies and overall operational quality. One component of the presently described system is a Workflow and Information Systems re-Engineering (“W.I.S.E.”) Change Platform which integrates services from a Clinical Context (“CC”) engine, an Electronic Data Integration and Transformation (“EDiiT”) engine, a Knowledge Management System with Vocabulary services (“KMS”), and a Virtual Hospital Visualization, Simulation, and Analysis (“Virtual Hospital VSA”) tool. As described in detail below, the present system facilitates individual and/or organizational change through various mechanisms including, simulations or serious games (e.g., games based training), decision support systems, process improvement and/or workflow re-design, information system lifecycle management, business analytics and intelligence, and knowledge management (e.g., discovery, acquisition, engineering, and dissemination).

The features of the system and related methods of this disclosure, and the manner of attaining them, will become more apparent and the disclosure itself will be better understood by reference to the following description of embodiments taken in conjunction with the accompanying drawings.

### BRIEF DESCRIPTION OF THE DRAWINGS

FIG. 1 shows an event performer matrix.

FIG. 2 is a process model diagram.

FIG. 3 depicts a combined model of an outpatient encounter.

FIGS. 4-6 depict screenshots of a component of the system.

FIG. 7A, FIG. 7B, FIG. 7C are an entity relationship diagram for the HL7 version 3 RIM.

FIG. 8A, FIG. 8B and FIG. 8C are a conceptual block diagram of components of an embodiment of the system of the present disclosure.

### DETAILED DESCRIPTION OF EMBODIMENTS OF THE DISCLOSURE

The embodiments disclosed below are not intended to be exhaustive or to limit the subject matter to the precise forms disclosed in the following detailed description. Rather, the embodiments are chosen and described so that others skilled in the art may utilize their teachings. More specifically, the present system is applicable to a wide variety of enterprises including research and development, manufacturing, and service delivery, to name a few. For the purpose of explaining the structure and operation of the system, an example case of a heath care delivery enterprise is used. The present disclosure is, of course, not intended to be limited to this particular application as will be readily apparent to those skilled in the art.

In general, the system and methods of the present disclosure address workflow management by first defining the current state of the enterprise activities. Table 1 lists examples of clinical workflows of patient care personnel.

TABLE 1
Archetypes of Clinical Workflow

| | |
|---|---|
| Admission | Assessments |
| Work-up | Medication Admin |
| Pre-Operative | Monitor/Respond |
| Intra-Operative | Hand-off/Sign-out |
| Post-Operative | RT/Vent Mgmt |
| Rounding | PT/OT/Rehab |
| Consult | Code |
| On-Call | Disease or |
| | Treatment |
| | Specific Pathways |
| Discharge | |

Next, the interplay between different systems within the enterprise is examined. In this phase, the system of the present disclosure provides tools and services for analyzing these activities and identifying optimal alternatives through simulations, “what-if?” and other analyses. Finally, the present system provides a framework for continuous improvement of the enterprise across all systems and throughout their respective lifecycles. This phase involves delivery of knowledge base, proven interventions, and implementation tool kits for affecting and sustaining appropriate changes. This holistic approach integrates accepted problem solving methodologies with a systems perspective to effectively and efficiently manage tightly coupled processes, products, and services throughout their entire life cycles.

The process of defining the current state of the enterprise activities generally entails a plurality of different information gathering and formatting techniques and technologies. Initially, the system of the present disclosure is connected to the various information systems of the enterprise to gain information about the relevant activities and states. These information systems may include patient, healthcare provider, and asset tracking, monitoring, communication and information systems. Such systems may include optical tracking systems (e.g., bar code readers), IR tracking systems, RF systems such as the Vocera® communication systems which, in addition to permitting wireless communication using the 802.11 standard, generate databases of information describing the communications (e.g., caller, time, location, content, etc.), RFID based tracking systems which identify people, role, and assets, provide time and location data, and in some instances status or other descriptive information about the use of a tracked item, and any of a variety of different information systems requiring manual entry of data. Furthermore, image, video, and other position sensing systems may be used to determine the location and activities of various persons, assets, and other entities. Various clinical and operational systems may also serve as a key source of data. In addition, control and administrative systems (e.g., financial, resource planning, scheduling, and supply management) and other meta-data systems (e.g., audit trails, log files, and usability data capture systems) may be used. Indeed, external information sources (e.g., weather or traffic reporting systems, etc.) may be linked to the present system. Finally, various manual methods of data collection (e.g. time-motion studies, ethnographic inquiry) may be employed and captured digitally (structured, semi-structured, and unstructured) to supplement the above data. Commercial, open source, service oriented architecture, and/or open standard interfaces are available to provide the above-described connectivity, and may be implemented accord to conventional techniques well-understood by those skilled in the art.

Once the present system is operationally coupled to these information systems, various methods of information gain are applied to the data including terminology and context mapping, record linkage, association rule mining, and probabilistic inference using Apriori knowledge (e.g., national guidelines, local best practices, and current state model predictions). This information gain need not be sequential and in certain embodiments is iterative. A general example is mapping some data in structured records to a given terminology, linking several records, determining the context of the records, mining an association or relationship to additional records, inferring the probability of missing information or relationships, and then mapping these new concepts to additional terminologies.

An effective and usable knowledge base provided by the present system includes lexical, syntactical, and semantic integration of knowledge representations. In other words, the knowledge base uses universal (standardized) vocabulary organized in an established structure or organization that effectively communicates true meaning. During the information gain process for defining the current state of the enterprise activities, the present system employs vocabulary services for terminology mapping. This may include mapping local terminologies to established standards recommended by the Consolidated Health Informatics Initiative including SNOMED-CT (with ICD-9 cross-maps), LOINC, HL7/UCUM, RxNorm UNII's, and NDF-RT drug classes. In one embodiment, this may done using a combination of NLM's UMLS and MetaMap. In general, the system applies different meanings to certain data items based on a variety of different linguistic concepts such as lexical knowledge, syntax, semantics (i.e., the understanding of meanings), and pragmatics (i.e., the use of language in contextual situations). Such services may enable further and accurate machine understanding of the data. Standard, local, or proprietary vocabulary systems may be used (e.g., WordNet or EMR dictionary).

Similar to applying terminology mapping for the data and records, the system will associate the context of the data to appropriate contextual properties (e.g., role, environment, activity). Context application may use defined or derived contextual nomenclature. The system may perform vocabulary and contextual information services separately or concurrently. Context refers to the relevant constraints, conditions, or other qualifiers of the situation or event represented by the data or record.

Furthermore, the system links records or data items based on selected or derived linkage identifiers. For example, many records in health care environments are linked by patient using name, gender, date-of-birth, social security number, and medical record number. Moreover, patients may be linked with healthcare providers, assets, locations, times, activities, etc. The associations between the data may, in one embodiment, be accomplished using probabilistic linkage technology such as iterative or estimation techniques (e.g., expectation-maximization algorithms). The system thus provides a means by which to link and co-model clinical information or medical actions (e.g., disease state, treatments, diagnostics) and physical workflow (e.g., task, time, motion, person, role).

The gathered, linked data may then be used to perform association rule mining to identify characteristics of the monitored activities not otherwise apparent from the individual data items. This generally includes considering the physical and clinical context of a set of linked data to infer additional information about that or the entities involved. Conventional rule mining is performed, for example, in diagnosis association groupings which may include data relating to disease, symptoms, location, findings, assessments, tests procedure, treatments, therapies, medications, risk factors, and complications. In the context of the present system, such rule mining may include, for example, association of patients with procedures such as determining that a patient is likely receiving a certain medical procedure because the patient is in the location where such procedures are performed and accompanied by a healthcare provider who performs the procedure, and/or the patient has an apparent clinical necessity for such procedure. Conventional rule mining is described in Algorithms for Association Rule Mining—A General Survey and Comparison, published in SIGKDD Explorations, July 2000, Vol. 2, Issue 1, beginning at page 58, the entire contents of which is hereby expressly incorporated herein by reference.

The present system uses the above-described connectivity and resulting information gain to generate, over time, a highly detailed model of the enterprise's activities. Generally, the longer the period of information gain, the more accurate the model. In one embodiment of the present system, the information gain extends over a period of at least six months to two years.

Use of the model resulting from the information gain stage may begin with defining a set of parameters for the various processes being studied wherein the parameters specify “how things should be done generally.” These parameters may be derived from experts or various other relevant knowledge sources, which are independent of the actual information gained from the enterprise (i.e., Apriori knowledge). The Apriori knowledge sources may include standard or commercial knowledge such as generally accepted clinical pathways and guidelines, known workflows, or other sources related to the activities of the enterprise. Some existing knowledge sources include the Veterans Administration's National Drug File-Reference Terminology, AHRQ's National Quality Measures and Guidelines Clearing House's and Health Care Innovations Exchange Website, Veterans Health Administration's Guidelines, and American College of Surgery's National Surgery Quality Improvement Program Guidelines. The resulting model constitutes a preliminary estimation of how the subject processes or workflows “should be done generally.”

Next, using conventional workflow analyses, the subject processes are characterized to generate a preliminary estimation of “how things seem to be done.” This may include a walk through with time-motion studies, task analysis, ethnographic inquiries, process mapping, and use of general data mining, visualization and business intelligence tools such as tools provided by Spotfire and Weka. It should be understood that this step is not required in all embodiments, or at least not occurring in this order. This step may be performed in an iterative manner.

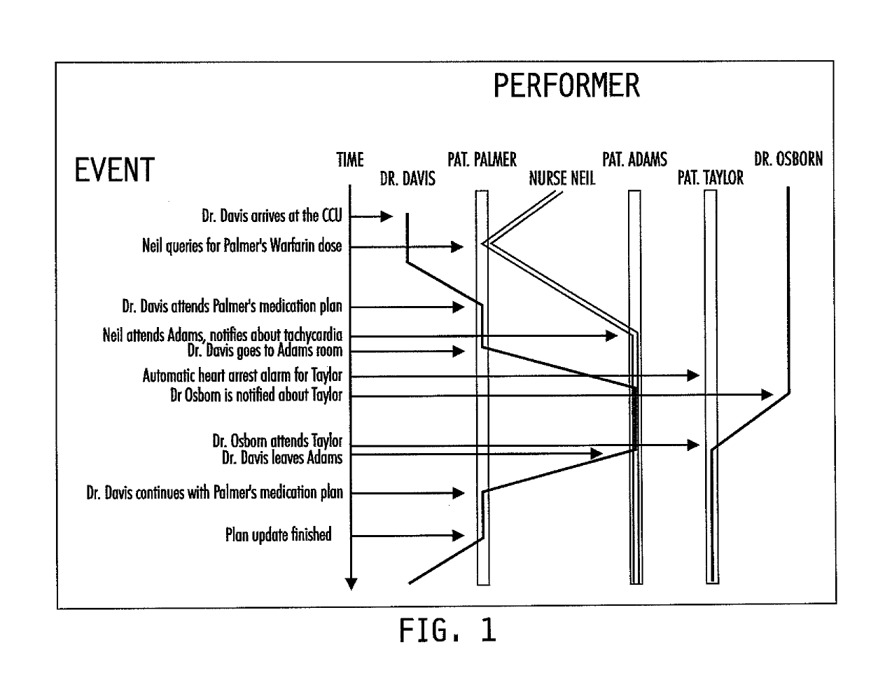

FIG. 1 shows event performer matrices, which is a methodology and tool for determining critical path and timing for human intensive workflows. It can determine optimal critical path as well as constraints or contingencies.

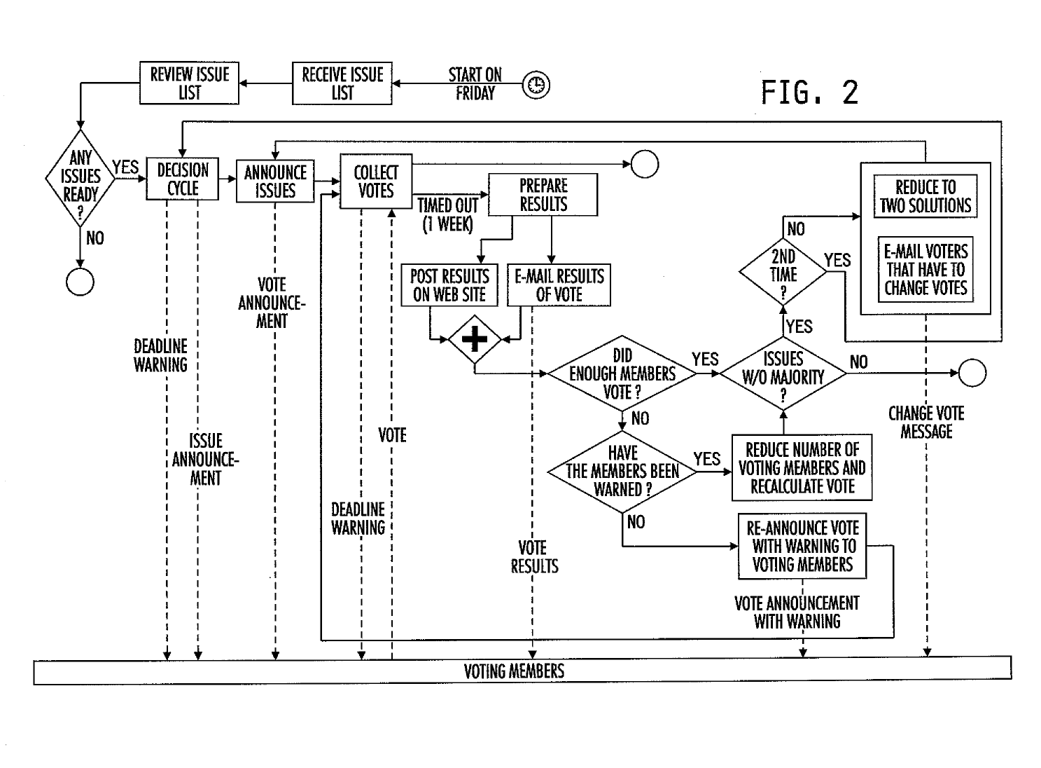

FIG. 2 shows an example of process modeling and “what if?” analysis. Formal process modeling allows for tight control over workflow, decision support, and formal application of “what if?” analysis. Process Modeling is used for delivering business rules for decision support.

Then, the preliminary estimates of “how things should be done generally” and “how things seem to be done” may be used to create a model using the above-described information gain. This use of the preliminary estimates to create the working model provides the starting point for determining “how things are really being done.” Various techniques are employed in the process of arriving at an accurate model for the subject processes of the enterprise. As the operations of the enterprise likely include concurrent behaviors of entities in distributed systems, petri net mathematics may be used to refine the model. Additionally, stochastic modeling techniques may be applied to the model to estimate probability distributions of the process outcomes by introducing random variations (or permitting them to occur naturally) over time. Similarly, repeated random sampling using Monte Carlo algorithms may also be employed by the system to estimate the behaviors of the various resources involved in the subject processes. Additionally, the system may transform the value-added information into a data model that co-models operational and clinical data with workflow and guideline knowledge to perform these analyses (see, for example, Web Services Business Process Execution Language Version 2.0 OASIS Standard 11 Apr. 2007 (http://docs.oasis-open.org/wsbpel/2.0/OS/wsbpel-v2.0-OS.pdf) or Conceptual alignment of electronic health record data with guideline and workflow knowledge, G. Schadow, D. C. Russler, C. J. McDonald—International Journal of Medical Informatics, 2001 (64) 259-274, the entire disclosures of which are hereby expressly incorporated herein by reference). Other machine learning algorithms and techniques may be employed as well (e.g. Hierarchical Temporal Memory models). Finally, a combinatorial optimization and analysis process can be used to determine the best method to model information gaps and assign best alternatives or derivatives (e.g., weighted combinations of modeled variables). In this process, all of the above-described techniques may be used concurrently and iteratively.

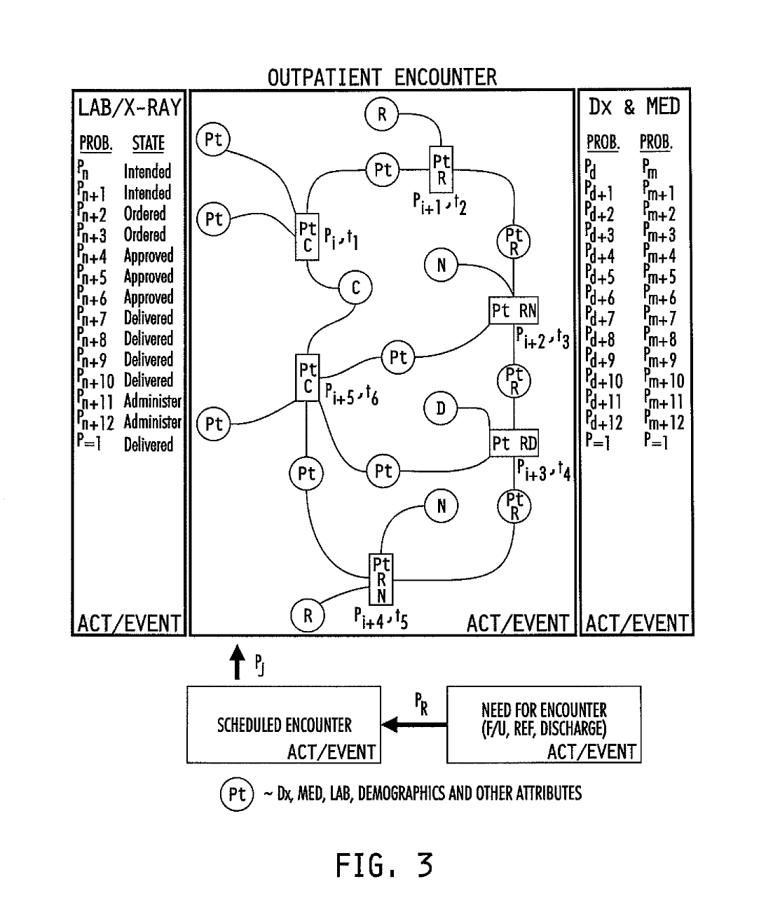

FIG. 3 shows a rough general example of the workflow related to an outpatient clinical encounter and related activities using a combined model including petri net mathematics, probabilistic techniques, and the contextual v3 RIM.

One tool for further refining the model is by providing feedback to the current state of the model definition through simulation or gaming. As is further described below, simulations provide other benefits (e.g., training, etc.), but in this context, the simulations permit the user to generate new enterprise information in an artificial (or virtual) environment using previously gathered enterprise information. More specifically, users may interact with a virtual or mixed reality game environment built with actual data and information derived above for the particular enterprise. The game may require users to participate in certain workflows, thereby introducing variations in workflow input from the user as opposed to from random or machine predicted. Using techniques mentioned above, the model predicts the outcomes resulting from the user's interaction. These predictions may be treated as actual enterprise information and used as feedback to the current state definition. This facilitates a means by which to derive workflow, domain, or local knowledge from human actors and integrate it into the current state model.

Use of serious games has other affects on workflow. For example, in healthcare it can affect the both caregivers and patients. Caregivers can have their knowledge of how workflow should be implemented upgraded to the current thinking or adjusted to fit the norm. Patients can be taught how to affect their personal care and the workflow that is involved in doing that themselves, as well as the consequences of inappropriate flow. Serious games provide the means to train people with a more engaging workflow context to the learning and the outcomes. By having people play games you can get them engaged right away in the goals of the competition, one that can be directly tied to their behavior.

As should be apparent from the foregoing, repeated simulations of various aspects of a process being studied not only provide valuable learning for the user, they permit exploration of the process through input modification and variation, which thereby permits rapid, reliable model refinement to converge on a true representation of “how things are really being done.” This exploration may be characterized as “what if?” analysis, wherein human inputs are provided to characterize likely outputs. Of course, computer algorithms may also provide the input variations. Where that is the case, the personnel training aspect of the simulations is absent, but the workflow exploration and characterization may be exceptionally comprehensive. Theoretically, algorithms may be provided to affect arbitrarily small adjustments to every variable for every process, permitting the system to automatically exhaust the possible behaviors of the processes being studied to determine the optimum workflow requirements or best practices. Of course, combinations of human and computer generated process variations may be provided as inputs as well. As the model is refined through these activities, the current state of the enterprise is updated to reflect “how things should be done in this particular enterprise.”

It should also be understood that in reality, changes to enterprise processes or workflows occur organically. Hospital administrators, for example, may determine that a certain step is a process should be altered. These real life modifications (as contrasted with simulated modifications) are automatically incorporated into the model through the information gain phase described above. In this manner, the model tracks the evolution of the enterprise. However, there can also be need for rapid modification as policy changes occur such as reimbursement compliance credential rules, legal requirements, etc. When this need arises, the system facilitates direct, immediate manipulation of workflow and outcome variables to reflect the desired, sudden modification.

An outgrowth of the pervasive connectivity and knowledge base of the system is its ability to provide Clinical Decision Support (CDS) to individuals or assets in the enterprise or to other information systems, a service that is widely regarded as directly impacting patient safety and quality of delivered care. CDS includes, among other things, alerts and clinical reminders, diagnostic support, adverse event monitoring, quality and safety reporting, information display, guidelines, interaction checking, as well as default (standing), recommended, and corollary orders. For example, upon identifying a nurse with unwashed hands through the information gain phase (conventional systems are available for detecting use of hand washing stations), the system may issue an instruction to the nurse to wash his or her hands through the existing communication infrastructure (e.g., pager, Vocera® device, cell phone, nurse call station, etc.). The system may further send a notification to the nurse's supervisor of the nurse's non-compliance with the enterprise hand washing protocol. In the process of providing such decision support, the system may leverage its context awareness to tailor its intervention. For example, the preferences of health care providers may be taken into account to determine whether to send a notification by pager or to more passively notify the provider by populating a report for the provider's subsequent review. Of course, the context of the support criticality may override the provider preferences. For example, even providers who dislike direct contact notifications may receive such notifications for highly critical support situations (e.g., notifications that a patient is about to be administered a drug to which the patient is allergic).

Additionally, the system may determine that the existing infrastructure does not support notification of the individuals needing decision support, and generate a report for use by administrators in deciding to invest in such infrastructure. As a further extension, it should be understood that the system may be configured to automatically impose real time adaptations to itself or interfaced systems based on the needs it identifies through the workflow analysis described above. For example, the system may identify though use that separate information systems should be in communication with one another (i.e., as opposed to using a human surrogate) to improve a particular workflow. By configuring the system with the proper network infrastructure, the system itself may establish the desired communication link to facilitate the improvement.

There are numerous forms and methods of clinical decision support that may be administered prospectively (standing orders), at point and time of care (e.g. CPOE, BCMA), or even retrospectively (e.g. Adverse Event Detection, Pay for Performance). While the trigger event is likely different, whether applied to an individual patient (or provider) or to a given cohort prospectively or retrospectively, the foundational knowledge base for the decision logic should be essentially the same. In other words, the knowledge to monitor HbA1C in a diabetic patient at a given time interval can be used to send scheduled lab visits to a patient, launch a clinical reminder when the provider renews diabetic supply orders, or report compliance rates to quality compliance officers. This is one advantage of the HL7 version 3 Reference Information Model (“v3 RIM”) which is further described herein. Medical knowledge, clinical workflow information, and clinical data are modeled as the same data entity with a simple state change represented in the Act moodCode (e.g., recommended, planned, scheduled, performed, resulted). Therefore, the same logic can be used regardless of the timing or method of intervention to be employed, effectively decoupling the general knowledge found in the present system from the applications that use such knowledge.

Currently, most decision support is integrated at various points within the ordering process (particularly for medications): at time of entry (Computerized Provider Order Entry or CPOE); at the point of order receipt and processing (Pharmacy Data Transaction System or PDTS); and at the point of order administration (Bar Code Medication Administration or BCMA and Medication Administration Record or eMAR). This closed loop approach is designed to ensure appropriate care and provide satisfactory redundancy at crucial provider interactions in the process (e.g., physician, pharmacy, nurse).

As described herein, the system of the present disclosure provides the primary features needed for effective CDS delivery: 1) an accurate, trusted, and manageable knowledge base; and 2) an efficacious, user-friendly, and configurable means to integrate decision support tools into clinical workflow and cognitive tasks.

For medication decision support, the following list describes some of the functions the CDS delivery components of the present system may provide, depending upon the embodiment:

A) Medication reconciliation, which may include comparing medication Acts (e.g., Ordered, Dispensed) based on therapeutic agent (active ingredient, UNII from SPL), drug class (from NDF-RT) and dose (sig), detecting transitions in care such as Admit, Discharge, Transfer as events to trigger comparison, and reporting results using specified protocol from alternative standards (API, WSDL, RPC, Arden Syntax).

B) Drug contraindication screening and adverse event detection, which may include importing DailyMed's v3 RIM based SPL in XML format from FDA website or alternate public or private medication knowledge source, extracting SPL defined attributes including indications, conditions of use (patient population, tests for monitoring, and adjunctive treatment), limitations of use (e.g., renal function) and contraindications (lab values, medications, demographics), adverse events and side effects, as well as drug interactions, mapping host system patient data to appropriate coding system for SPL attributes, monitoring v3 RIM or other clinical messages for ICD-9/10 E-Codes, executing data comparison (decision support) logic such as presence of contraindication or E-code, determining and providing alternate outputs for event detection such as Adverse Event (AE) forms, Alerts, most recent laboratory values or trends (e.g., drug levels, GFR, K, TSH, Cr), and change of order status (needs override).

C) Medication screening for drug duplication or therapeutic failure, which may include standardizing coding and classification of medications using SPL, RxNorm, and NDF-RT, executing data comparison for drugs, classes, and dosages (sig), and determining and providing alternate outputs for event detection as mentioned above.

D) Dosage checking, which may include, in addition to functions listed in C) above, including HL7/UCUM standardized units of measure, extracting dosing instructions from SPL or other knowledge sources, and identifying similar tools for managing calculations as part of the knowledge base.

E) Management of corollary orders, which may include extracting information from a knowledge base using SPL's conditions of use attribute (e.g., tests for monitoring) or other knowledge source, executing logic for identifying orders with known corollaries, and determining and providing alternate outputs for corollary order creation such as HL7 version 2 ORM or version 3 Act class Observation with an actMood Code of “recommended.”

F) Indication for medication, which may include mapping of problem terms to standardized terminology (SNOMED-CT and ICD-9).

G) Extraction of information from a knowledge base, which may include using SPL's indication attribute, Veterans Administration's National Drug File-Reference Terminology, AHRQ's National Quality Measures and Guidelines Clearing House's and Health Care Innovations Exchange Website, Veterans Health Administration's Guidelines, the American College of Surgery's National Surgery Quality Improvement Program Guidelines, and other guideline knowledge sources.

As indicated above, the foundation for the system of the present disclosure is the Workflow and Information Systems re-Engineering (“W.I.S.E.”) Change Platform which integrates the CC engine, the EDiiT engine, the KMS, and the Virtual Hospital VSA tool. The EDiiT engine is a data integration and transformation tool that applies understanding (e.g., lexical and semantic) and transformation to a contextual data model that aligns and integrates clinical and operational data with workflow and medical knowledge representations. The CC engine facilitates the acquisition of context, the abstraction and understanding of contextual meaning (e.g., pragmatics), and the application of behavior based on recognized context. The KMS standardizes vocabulary terms across systems and establishes probabilistic, temporal, and semantic (meaningful) relationships between terms and concepts. The system may function in such a manner that the EDiiT engine, CC engine, and KMS are functionally or effectively a single system similar to HL7 Java SIG which is an implementation of the v3 RIM contextual data model that is capable of representing clinical and operational data, workflow and knowledge and providing integration and transformation services of electronic data to and from external systems. Further description is provided at aurora.regenstrief.org/javasig, the entire contents of which is hereby incorporated herein by reference. The Virtual Hospital VSA tool provides the user with a complete and accurate view of clinical operations. By leveraging the functionality of the aforementioned components with their clinical, administrative, and information system data, information, and knowledge, and by spatially tracking personnel and assets as described below, the Virtual Hospital VSA tool provides a useful view into the details of the daily activities within a hospital or healthcare setting. The Virtual Hospital VSA tool is used for defining and documenting workflow, performing analysis and re-engineering of processes and information systems, and training of personnel in these enhanced behaviors.

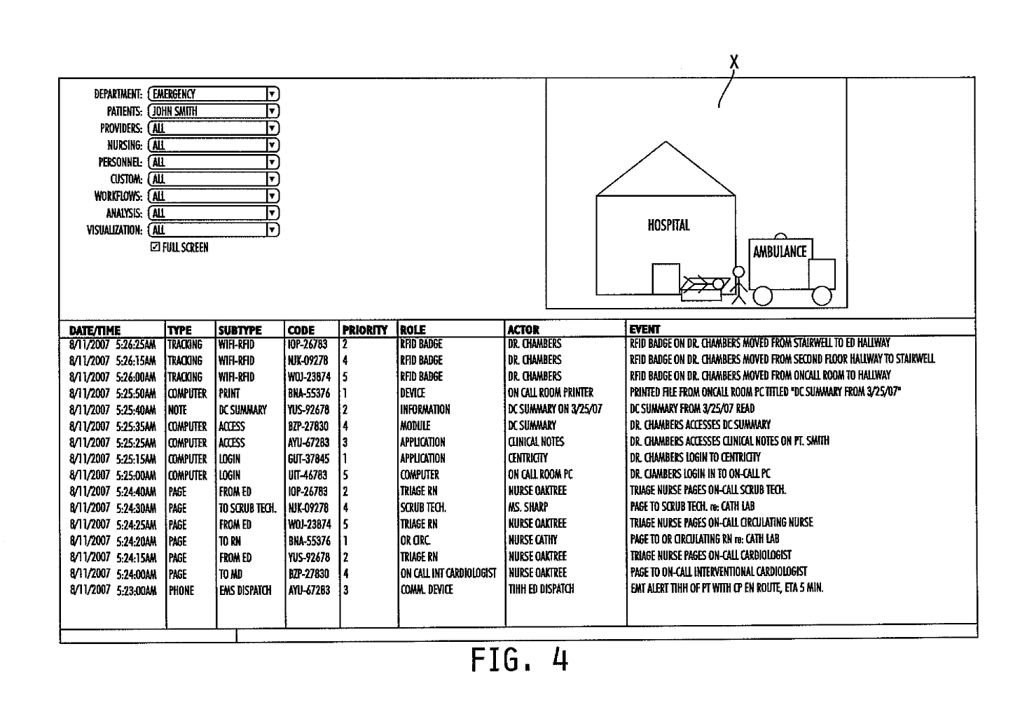

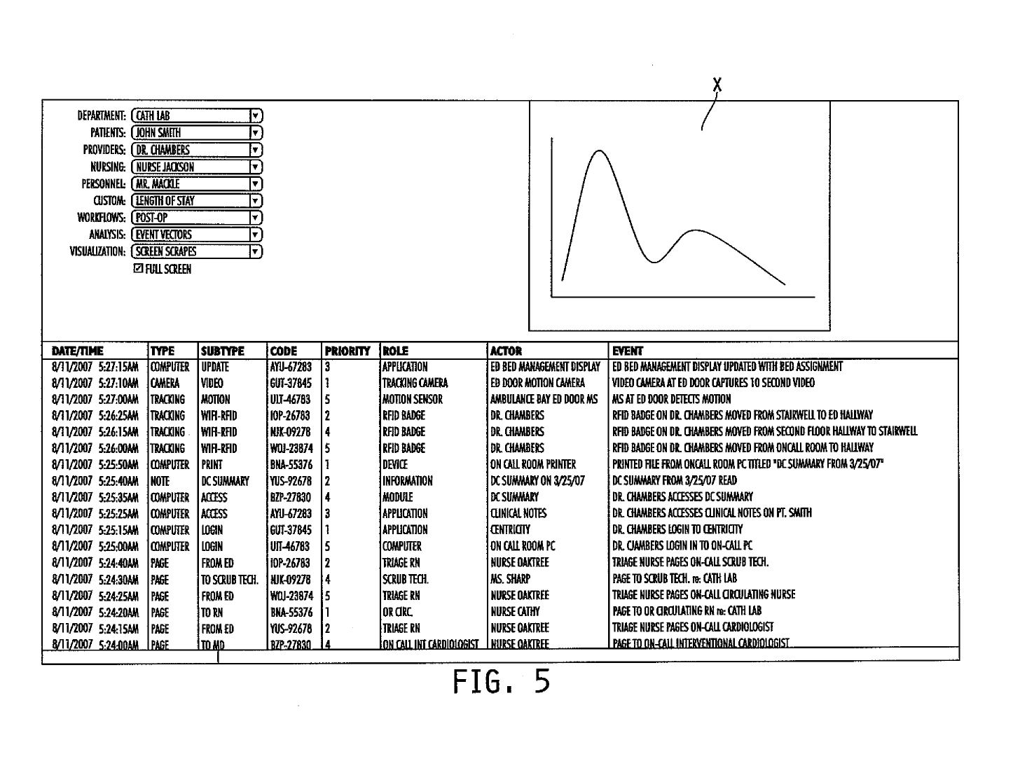

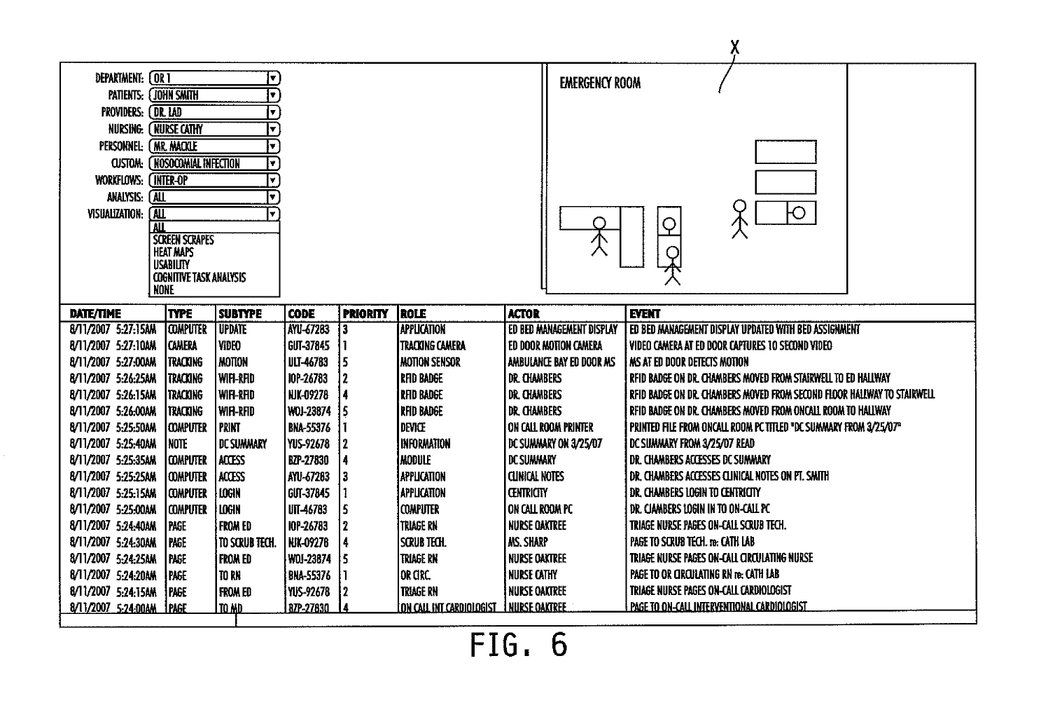

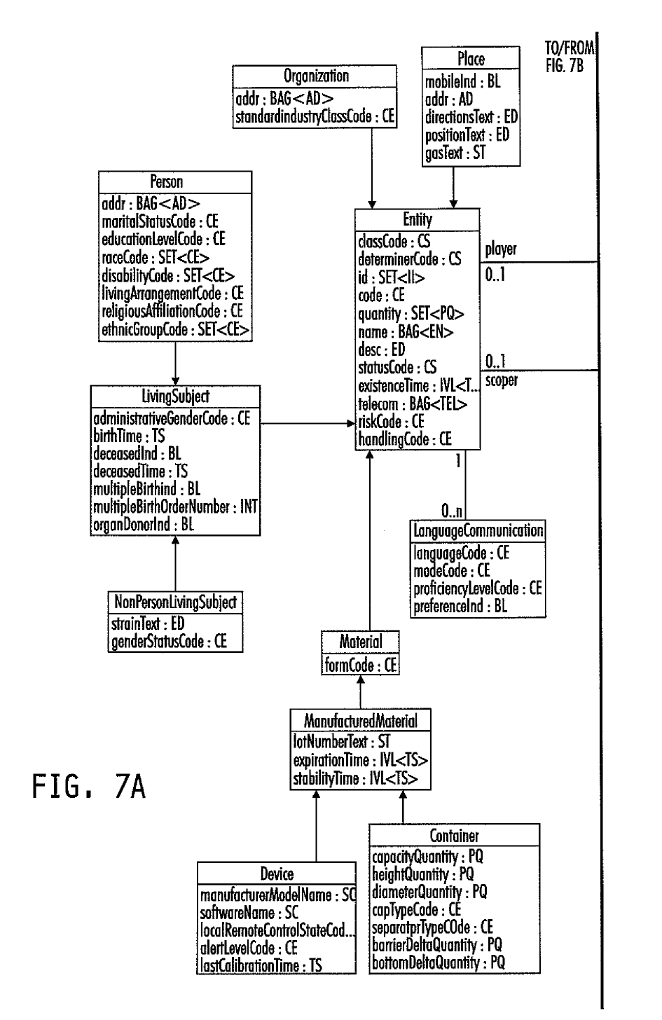

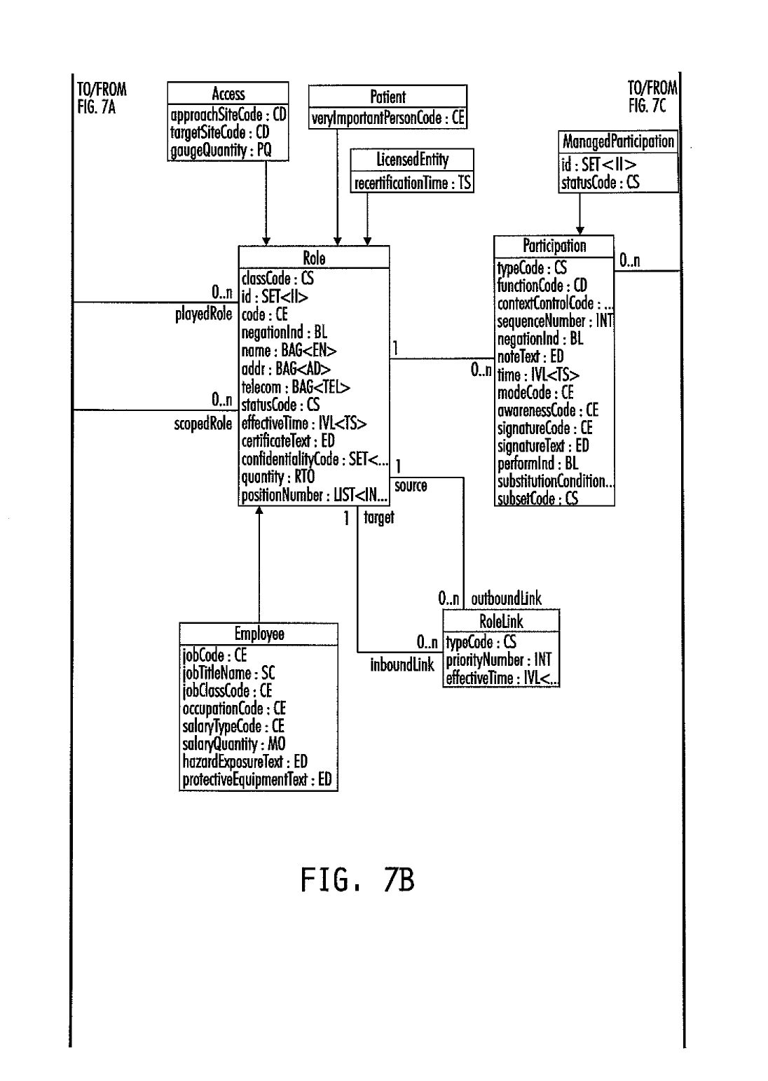

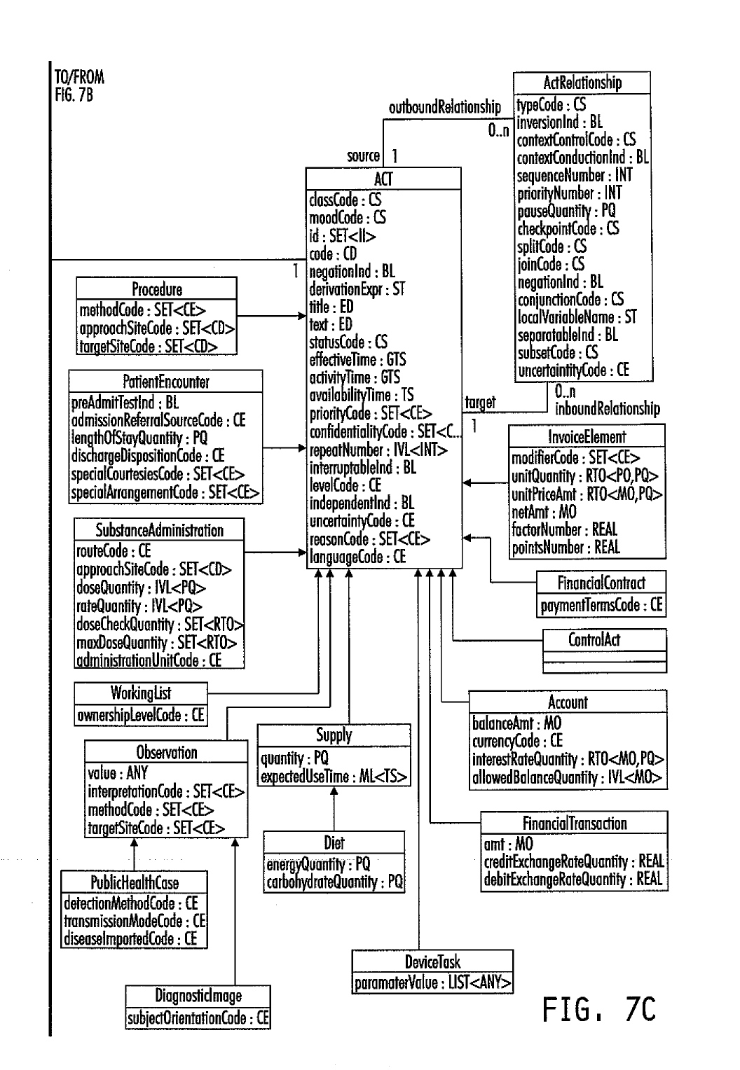

FIGS. 4-6 depict a part of an embodiment of a Virtual Hospital VSA according to the present disclosure. As events are depicted in window X of FIG. 4 (and FIG. 6), descriptive information is displayed in real time in the lower window of the screen. The lower window can be configured using the control buttons in the upper left corner of the screen. The control buttons also control the content displayed in window X (see FIG. 5 where an event vector (chart) has been selected for display).

Stated another way, the system of the present disclosure employs a holistic approach to solving problems with the aid of appropriate tools, technologies, and experts. First, the system of the present disclosure defines existing workflows (e.g., operation of emergency cardiac services, hospital borne/spread infections, staff scheduling, operating room (OR) patient flow) with minimally invasive techniques and technology embedded in the W.I.S.E. Change Platform and its associated components. This enables minimal disruption to their current workflows and results in a highly defined “current state” from which to develop problem resolution. This “current state” typically represents at least the previous few months of fairly detailed physical, operational, and clinical workflow and information tasks and events; not merely a generalized, high level snapshot of an afternoon walk-through. For use in the system, workflow knowledge can be pre-defined, derived, discovered, and/or represented in a knowledge base. Methods for acquiring workflow knowledge include ethnographic methods such as contextual interview and time-motion studies as well as statistical analysis with Petri-Nets, Markov Models, and Agent-based modeling. Each state and transition in a Petri-Net can be represented as an Act or ActRelationship from the v3 RIM, which is described in publicly available documents provided by www.hl7.org. such as the document found at www.hl7.org/Library/data-model/RIM/C30204/rim.htm and HL7 Reference Information Model Compendium (RIM version 2.01) available at www.hl7.org.au/HL7-V3-Resrcs.html, the entire disclosures of which is hereby expressly incorporated herein by reference.

As indicated above, the system of the present disclosure performs measurement, analysis and optimization of local best practices including process, policy, training, and implementation of ideal information tools and technology. This facilitates identification of clinically critical and high return on investment opportunities. The knowledge gained allows for the re-engineering of workflows, processes, information tools and training to move the enterprise closer to the desired or discovered outcomes in key clinical and operational areas.

The system of the present disclosure also provides, in certain embodiments, the tools, services, and expertise to enable enterprise administrators to continually improve and control these endeavors, as well as to maintain, manage, share and customize the contents of the knowledge base. In one embodiment, the system includes a means for keeping it current with the accepted knowledge sources mentioned herein. For instance, as a new drug, indication, drug interaction, etc. is added to the FDA's DailyMed (SPL), the knowledge base through its connectivity to external user information systems, maintains these updates as well. The system also includes the ability to acquire, represent, analyze, create, test, and disseminate new and/or local knowledge. As further described herein, this function may include use of artificial intelligence algorithms not limited to Bayesian belief networks, inference detection, stochastic modeling, agent based modeling, Fourier transforms, and other machine learning methodologies to detect potential knowledge. As discussed herein with reference to the simulation features of the present system, the system's features for analyzing, modifying or discovering knowledge inferences may include a mechanism for an expert to interact with advanced data mining and visualization tools.

Such visualization tools include the Virtual Hospital VSA tool to observe real-time work and information flow and perform basic and advanced analyses such as “What if?” scenarios. The above-mentioned services and expertise may include process engineering (simulation, modeling, as well as Lean Six Sigma), clinical informatics (design of and interface with clinical information systems), and statistical analysis (stochastic, Bayesian, and multivariate analysis of indicators and outcomes).

In a subsequent phase of operation of the system of the present disclosure, the W.I.S.E. Change Platform is used to install, train and begin to monitor outcomes. The Virtual Hospital VSA tool, for example, can function as an advanced training tool. The more visually realistic and personally relevant the training environment (a virtual representation of a user's own clinical environment), the more effectively and quickly the user is able to learn and adopt the presented best practices as well as react to rare events. A high level of realism tightly couples training with process improvement and the system as a whole as well as significantly impacting retention and comprehension. Using Problem-Based Embedded Training as is further described herein, personnel are often unaware to whether they are training or performing or even both. Finally, feedback from the training can provide insight into process challenges, complexity, or opportunities.

The Virtual Hospital VSA tool is an end-user application that provides direct value to the user by enabling a direct view into clinical operations. It is a simulated visual view into hospital operations and clinical workflow. The Virtual Hospital VSA tool utilizes the W.I.S.E. Change Platform to transform various sources of data into a virtual model for direct visualization and analysis. The Virtual Hospital VSA tool aids administrative personnel whose responsibilities include quality improvement/enhancement as well as hospital operations such as staffing, scheduling, policy, training, and other organizational activities. By spatially tracking personnel and assets in combination with various clinical, administrative, and information system data, this tool provides a unique and high-value view into the details of the daily activities within a hospital setting.

### Electronic Data Interface, Integration and Transformation (EDiiT) Engine:

Gathering all of the data that is necessary for delivering quality information that is contextually relevant and in a form needed for making informed decisions is a difficult challenge which grows daily as new systems are implemented and new technologies and procedures are developed for the enterprise. Most of these systems have their own proprietary data formats and require software to extract the information that is relevant to the task at hand. The EDiiT engine provides this functionality across the enterprise with a highly developed standard that permits users and systems to communicate efficiently.

The EDiiT engine is a data interface, integration and transformation tool that enables the exchange of different types of electronic data. It also allows data to be understood (e.g., lexically and semantically) and transformed to appropriate formats (e.g., data model or syntax). In one embodiment of the disclosure, the v3 RIM is employed. As depicted in FIG. 4, v3 RIM combines clinical data, workflow information, and medical knowledge in a common, contextual data model. This model is based on the Medical Action framework which states that all medical information is a state representation of a medical activity with knowledge being what should be done, workflow being what is presently being done, and clinical data representing a completed or past activity.

As known to those skilled in the art, this is a contextual data model that aligns and integrates clinical data with workflow and medical knowledge representations (see, for example, Conceptual alignment of electronic health record data with guideline and workflow knowledge, G. Schadow, D. C. Russler, C. J. McDonald—International Journal of Medical Informatics, 2001 (64) 259-274, the entire disclosure of which is hereby expressly incorporated herein by reference). Transforming existing data, information, and knowledge into this data model enables its utilization for clinical practice, workflow analysis and optimization, decision support (individually or institutionally), and other use cases simultaneously and with the value added information from these other axes.

Knowledge Management System with Vocabulary Services (“KMS”):

Being aware of the volume, diversity, and complexity of information that exists in the health care enterprise is a daunting task for all health care professionals. It is even more difficult for knowledge based information systems providing quality data and supporting key processes to allow users and subordinate systems to get information quickly and accurately. The fundamental challenges are in defining, exchanging, and managing lexical and contextual data so that they share common semantics (true meaning) across all systems—computer and human.

The KMS works hand in hand with EDiiT engine. It discovers, creates, represents/labels, modifies, distributes and/or otherwise manages knowledge (clinical, workflow, and operational) for reuse, awareness and learning by both humans and machines. It is the repository and management local for vocabulary/terminologies, relationships, and rules for different entities, processes, functionalities, actions, and tasks. Vocabulary services of the KMS enable lexical and semantic meaning of data and information across the enterprise and between multiple systems. Thus, it facilitates “computer understanding” for the utilization of various advanced algorithms (e.g., Al, Bayesian Networks, Markoff Models) for high value tasks such as knowledge and opportunity discovery as well as the various decision support interventions mentioned above. A shared clinical and operational meaning is also useful for the effective utilization of numerous measurement, analysis, and optimization tools.

One example of a conventional knowledge management system (without a vocabulary system) that could readily be adapted for use in the present system is the KMS suite sold by CSW Group as well as the other systems mentioned herein.

### Clinical Context (“CC”) Engine:

To optimize performance, health care enterprises must make sense of the massive amounts of data and information that exists. Much of the complexity of this task is in tying patient, location, time and provider constrained information together with a disease/treatment clinical pathway or other knowledge source to make the best decision. Filtering through massive silos of disparate data to acquire the correct information for making a time-constrained and critical decision for a patient or a process level decision from a higher level is challenging at best.

In order for data to become high quality, value-added information, the data consumer (human or computer) should, at a minimum, be made aware of the context in which the data was captured, processed, stored, and presented. Context includes the relative constraints, situational influences, relevant inferences, and descriptive conditions under which a datum is acquired, stored, and used. It is the information about the circumstances under which a system is able to operate and, based on rules and/or an intelligent stimulus, react accordingly. Context aware tools are concerned with the acquisition of context (e.g., using sensors to perceive a situation), the abstraction and understanding of context (e.g., matching a perceived sensory stimulus to a context), and application of behavior based on the recognized context (e.g., triggering actions based on context).

Clinical data can have very different meanings based on its method of acquisition, patient “specimen”, environmental conditions, and clinical status of a patient. For example, temperature and fever in a post-operative patient can infer very different clinical situations based on the degree of temperature change, the time since the operation, the overall health of the patient, and the patient's age. In another example, a clinical reminder (a decision support alert) was sent to a fifty year old woman who had not had a screening mammogram in over two years (a very important breast cancer prevention metric). However, the system did not take into account that the woman was currently intubated and in the ICU. In the context of a very ill and possibly terminal patient, a screening test for primary prevention is not only clinically irrelevant, it would be operationally disruptive and even potentially risky for the patient. Perhaps the most significant consequence of such system failures has been that physicians often ignore such decision support interventions and even form very rigid biases about or against this form of technology in clinical practice. In general, patients' demographics, medical condition, active therapies, past medical history, current disease and physiological state, and stage of treatment all have significant impact on medical decision making and subsequently resource requirements, clinical pathways, clinician workflows, and even administrative and practice management processes.

Organizations face continuous and unprecedented changes in their respective business environments. Such disturbances and perturbations of business routines must be reflected within the business processes in the sense that processes need to be able to adapt to such change. Context provides fundamental information for sensing, processing, and reacting to various stimuli—the fundamental functions of an adaptive system. In as much, context has been recognized as being valuable to appropriately flexible and even necessarily adaptive processes as throughout the entire life-cycle of business process management and information system development.

The CC engine is a technology that takes relevant contextual data and discovers pertinent, value-added information for users or other applications in both physical and digital environments. Other conceptual frameworks such as clinical data mining, clinical workflow, and medical knowledge management are also likely users of this type of information. It takes these disparate types of information and analyzes, merges and distributes that information to the relevant entities.

The contextual information includes, but is not limited to user, environmental, and purely clinical axes. The classic contextual dimensions include: role, mode, (co)-location, visual data, bio-physiological state, social environment/relationships, tasks, priorities, modalities, qualifications/credentials, infrastructure/resources, physical properties (e.g. CAD), environmental conditions, etc. Additionally, context in a clinical setting includes dimensions and variables pertinent to this specific domain. This may include:
Patient—Demographics, Diseases, Therapeutics, Test Results, PMHx/SHx, Current Symptoms, Location, etc.
Provider—Role (MD, PA, NP, RN, Admit, Attending), Specialty, Experience, Workflow, Location, etc.
Setting—ED, ICU, Ward, Ambulatory, On-call, Cross-coverage, etc.
Workflow/Mode—Admission, Work-up, Pre-Operative, Intra-operative, Post-operative, Rounding, Consult, On-Call, Discharge, Hand-off/Sign-out, etc.

While the discussion above provides overview, functionality, and application information relating to the system and methods according to the present disclosure for the purpose of enabling one of ordinary skill in the art to practice the claimed invention(s), further implementation details are provided below to enhance the disclosure of certain features.

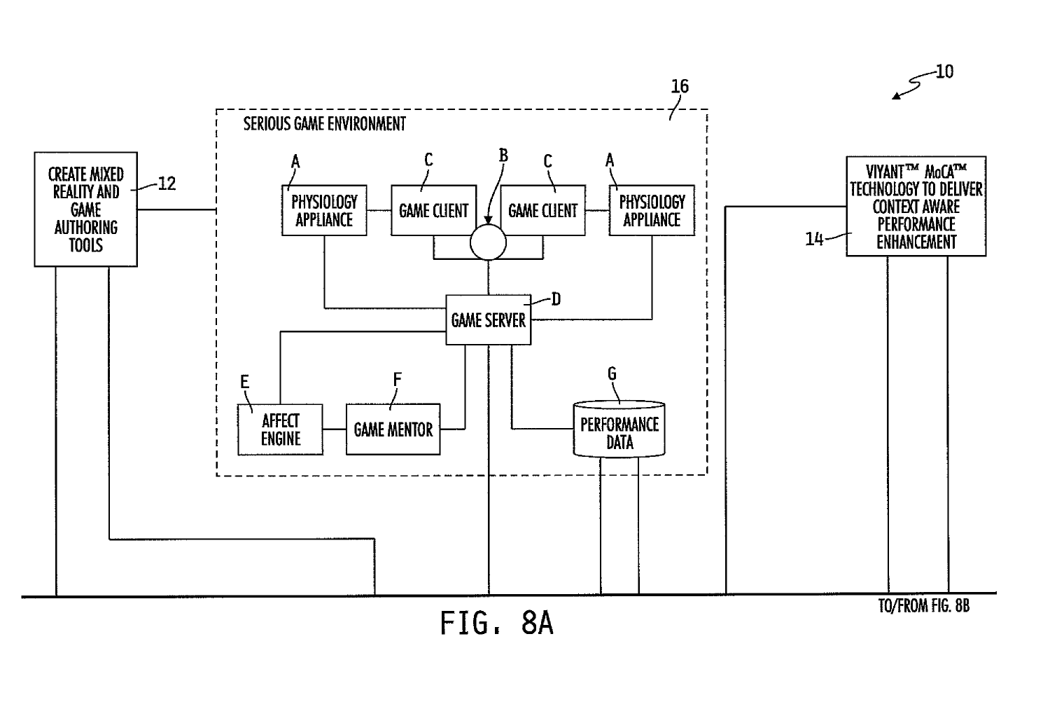

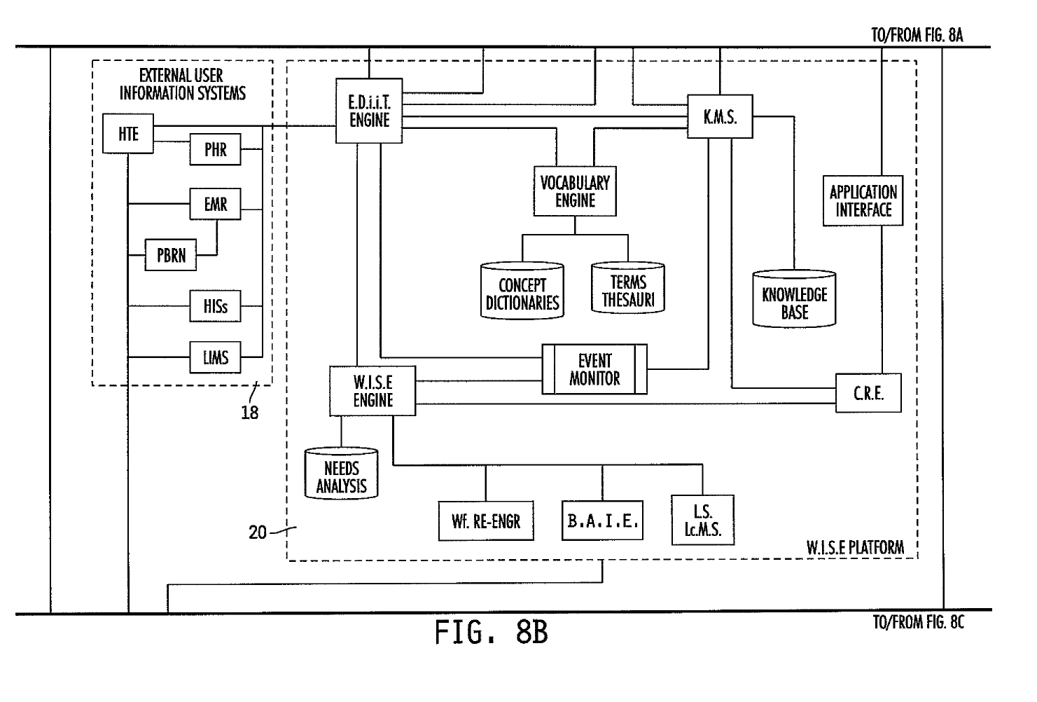

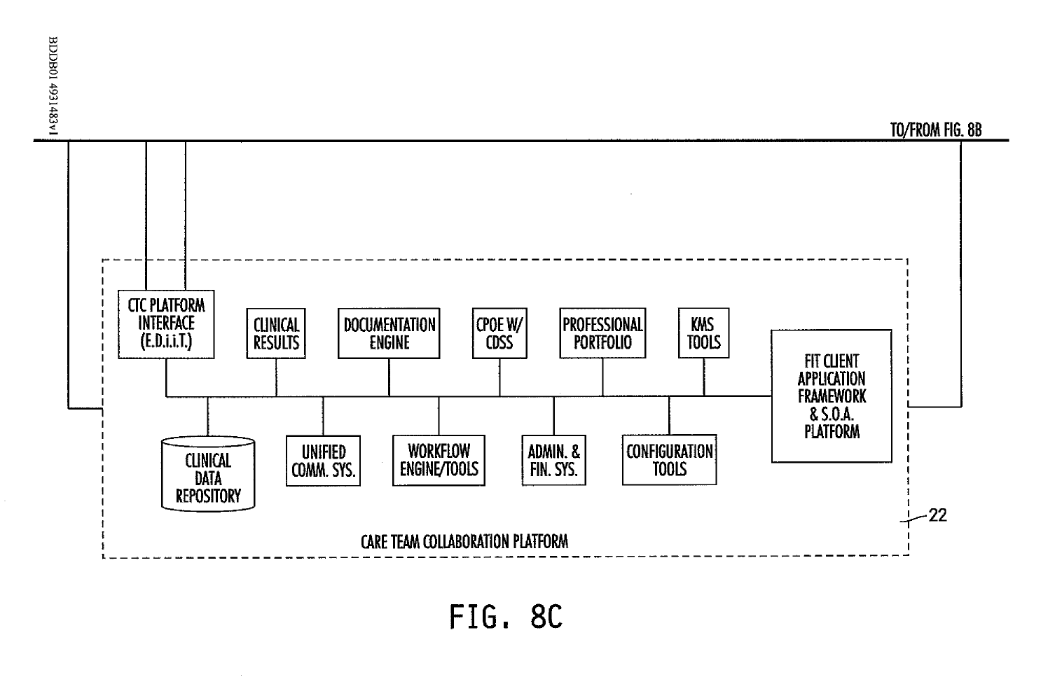

Referring now to FIG. 8A, FIG. 8B and FIG. 8C, a system 10 according to the present disclosure may include a variety of different components as shown. In general, system 10 may include a mixed reality and games authoring tool 12, a context awareness platform 14, a gaming environment 16, external user information systems, collectively referred to by the numeral 18, the W.I.S.E. Change Platform 20, and a Care Team Collaboration Platform 22. It should be understood that the Virtual Hospital VSA described above is an example of a gaming environment 16.

Authoring tool 12 is a mixed reality and video game authoring tool system which allows for the iterative development of mixed reality and video games by allowing for dynamic editing of mixed reality and video game environments. Thus, the parameters of the mixed reality or video game environment may be altered while a user is within a mixed reality or video game environment and the presentation refined in response to user interaction. In the context of system 10 as described herein, authoring tool 12 is used to design and develop the serious games (described more fully below) and describe the appropriate serious game environment needed to facilitate the desired learning, behavior modification, and/or desired result. A full description of the structure and operation of authoring tool 12 is provided in co-pending U.S. patent application Ser. No. 11/216,377, entitled “OBJECT ORIENTED MIXED REALITY AND VIDEO GAME AUTHORING TOOL SYSTEM AND METHOD” and filed on Aug. 31, 2005, the entire disclosure of which is hereby expressly incorporated herein by reference.

Context awareness platform 14 is a system that tracks the context of a user or object through software and hardware interfaces, both stationary and mobile. A commercial version of context awareness platform 14 is the Viyant™ product sold by Information In Place, Inc. and described at www.informationinplace.com. The tools within platform 14 gather and deliver contextual information to devices and provide data, audio and visual tools for collaboration and sharing. Platform 14 is designed to gather information from the user through graphical user interfaces, software that can infer information from rules engines and hardware devices that can provide information such as and not limited to location, biofeedback, equipment output, video and audio information, etc. Platform 14 gathers and disseminates contextual information to fixed and mobile platforms and tools for collaboration like video and image sharing, audio communication, and virtual whiteboards. Platform 14 further uses standard communications protocols to share and store data for use within the platform. The system uses standards for voice over internet protocols and video streaming and uses standard interfaces and databases to store and retrieve data as needed.

Gaming environment 16 includes physiology appliances 16A, a game engine 16B including game clients 16C and a game server 16D, an affect engine 16E, a game mentor 16F, and a performance database 16G. As shown, game clients 16C are functionally coupled to game server 16D, physiology appliances 16A are functionally coupled between game server 16D and game clients 16C, performance database 16G is functionally coupled to game server 16D, game mentor 16F is functionally coupled to game server 16D, and affect engine 16E is functionally coupled between game server 16D and game mentor 16F.

Physiology appliances 16A may be any device or software that either provides actual biophysiological data (e.g., feedback devices that monitor user heart rate, breathing, motion, visual tracking, affect expression, etc.) or simulates biological or physiological data of a user or other simulated entity for the purpose of affecting game play. The data is fed into the game through software that converts the data to a usable format for the game that has been designed to be affected by such data through affect engine 16E.

The game clients 16C of game engine 16B may be any appropriate client (e.g., software, hardware, or some combination) that enables the user to interface with and participate in the selected game. Standard clients and devices that exist in the marketplace may be incorporated into the game using techniques generally known to those of skill in the art. Game server 16D may similarly be any appropriate server device (or set of devices) that is in cooperative communication with game clients 16C to facilitate play of the selected game. While a server/client architecture is depicted, it should be understood that game engine 16B may in some embodiments include a single device for executing the selected game and providing user interface. Regardless of the embodiment selected, the game deployment should fit within the normal parameters of game deployment and be such that it is effective for the game and the outcomes desired.

Affect engine 16E is a tool which permits game mentor 16F to modify parameters or features of the selected game at any time from game set-up and through run time. These modifications affect the game dynamics and/or the player's emotional state to enhance the gaming experience and provide more effective training.

Game mentor 16F is an interface that permits a user, such as an instructor, who is monitoring the game to have access to affect engine 16E. This interface gives graphical user interface (GUI) elements that connect to event, actions or data within the game and allow the user to change some element of it. These items are designed into the game and made available as a tool for change so that the mentor can effect the player's experience. Additionally, certain tools within affect engine 16E will have associated rules that utilize physiology appliances 16A to apply changes to the player's experience. These may be threshold based and the GUI of game mentor 16F may allow for setting the thresholds and outcomes for the player.

Performance database 16G is an collection of data relating to one or more users and their previous actual or perceived gaming experiences. This data may be used during current or future game sessions to assess behavior changes or learning, or for research into future game designs and uses. The design of the game will dictate how this data is capture and used. Data of various types including the entire game interaction are saved to a database using conventional techniques. This data is designed to be accessed by various interfaces and systems during and after the game play.

External user information systems 18 includes the various external systems (i.e., communication, tracking, IS, etc.) mentioned above.

An example of a clinical event monitor, similar to the event monitor shown in FIG. 8A, FIG. 8B and FIG. 8C as part of W.I.S.E. Change Platform 20 is described in Design of a Clinical Event Monitor, Comput Biomed Res. 1996 Jun.; 29(3):194-221, by Hripcsak G, Clayton P D, Jenders R A, Cimino J J, and Johnson S B.

The following are examples of applications of the system according to the teachings provided herein:

Virtual Operating Room:

This application may involve modeling peri-operative (pre-, intra-, and post-) processes, workflows, and outcomes (clinical and operational). OR operations are perhaps the most intensive environments in terms of total patient care—clinicians (at least 2 MDs+3-4 staff), technology (instruments, devices, and pharmaceutics), and severity of relative morbidity. These service provided in OR applications are some of the most costly as well as greatest revenue generating services provided in healthcare. Much of the knowledge obtained and tools created in this setting may be translated to additional procedural care settings.

Virtual Radiology:

This application may involve modeling integrated health systems radiology services, processes, workflows, and outcomes. This includes registration and scheduling of inpatient and outpatient services for an integrated delivery model. This is also a significant revenue center with likewise significant operational costs.

Virtual Ward(s):

This application models clinical workflow, processes, and outcomes within general and specialty inpatient wards within the hospital. This is the setting for the greatest amount of patient care by patient hours and length of stay. This includes clinical and administrative activities within individual wards as well as across the enterprise. Specific applications include: 1) identifying, analyzing, alerting, and preventing the spread of hospital borne infections (a significant patient safety issue as well as a source of cost for the system); 2) acuity based scheduling (staffing based on patient needs); and 3) clinician sign-out (shift change and relevant information and task hand-offs).

Virtual ICU:

This application models clinical workflow, processes, and outcomes within intensive care wards within the hospital. This is the setting for the most intensive, non-operative patient care by total orders, nursing care per patient, disease severity, and costs. It is also a setting with a significant amount of data capture (automated and manual).

Situation Room:

This application provides a central hub of operational awareness for key administrators and physicians within an enterprise. It also provides appropriate views into current and historic clinical operations. It includes a “dashboard” design with highly configurable data/information elements and mechanisms of display.

Acuity Based Scheduling:

This application models staffing and scheduling based on predicted patient needs for maintaining quality of care and controlling variable costs. This exemplifies a use of clinical data and context to predict resource and operational needs as they continually change. This requires fairly detailed analysis of patient clinical requirements as well as personnel performance.

Infection Control:

This application provides features for identifying, analyzing, alerting, and preventing the spread of hospital borne infections. Identifying personnel and assets as possible carriers or sources of disease spread and invoking an appropriate intervention as quickly as possible can significantly reduce the incidence of such infections. This clearly has implications for quality of care, as well as length of stay and reduction of expense as many payers (including Medicare and Medicaid) are refusing to pay for care necessitated by a preventable adverse events such as hospital acquired infections.

Time to Reperfusion for Acute MI (Heart Attack):

This application provides features for capturing, analyzing, measuring, documenting, and improving activities related to the time it takes to get a patient from the ED door to blood flowing in coronary arteries. This is critical for patient survival and severity of subsequent disease. This is a key quality indicator for a heart hospital as well as any emergency department and can significantly impact operational measures such as length of stay.

Length of Stay (LOS):

This application provides features for capturing, analyzing, measuring, documenting, and improving activities related to the length of time a patient stays in an inpatient setting. This has implications for patient care as well as hospital reimbursement. Hospitals are typically paid by Diagnosis Related Groupings regardless of the time a patient spends in the hospital. The longer a patient stays in the hospital, the more likely they are to experience an adverse event such as hospital acquired infection, a medication error, or even a fall that can lead to serious morbidity.

Workflow Documentation and Analysis:

Inherent to the Virtual Hospital VSA tool is the ability to capture, model, and document workflow, a set of functions that provide the foundation for the above applications. Adding the capability to analyze the workflow information that is captured is a fundamental tool for process improvement (as well as decision-support interventions). Various analyses can be performed on normalized workflow and clinical data using established methods for such analysis (Petri-nets, actor/event matrices, process flow models, clinical outcomes analysis, etc.). “What If?” analyses provide tools for quality engineers to evaluate alternate workflows and present reasonable process candidates to front-line process improvement initiatives. Near real-time review of newly implemented interventions not only allows for risk minimization, but provides the content for lessons learned and various training modules.

Nurse Training:

The Virtual Hospital VSA tool also functions as a training tool for nursing staff heads, using a near real world environment. Training the nursing staff in basic patient care, as well as in re-engineered processes and workflows permits sustained operational performance enhancement. In addition, this training tool facilitates effective After Action Reviews similar to lessons learned from process improvement efforts.

Requirements for Clinical Information Systems:

Documented workflows and process improvement outcomes provide the foundation for information needed for the design of useful IT tools for clinical operations. From this knowledge base, the content for various design tools can be derived including use cases, personas, workflows, constraints, and requirements. This enables the organization to identify specific needs with their relative priorities values for information systems to be purchased, developed, or modified.

Comments on Provisional Application Ser. No. 60/948,924:

The following items refer to items depicted to FIG. 1 of the provisional application and provide, in certain instances, reference(s) to further, related description, all of which are hereby expressly incorporated herein by reference:

10.2
Conceptual alignment of electronic health record data with guideline and workflow knowledge, International Journal of Medical Informatics 64 (2001) 259-274
Adaptive Workflow Management in WorkSCo, Proceedings of the 16^{th} International Workshop on Database and Expert Systems Applications (DEXA'05). 1529-4188/05, 2005, IEEE
The Unified Service Action Model: Documentation for the clinical Area of the HL7 Reference Information Model, Regenstrief Institute for Health Care, 2000, Cleveland, Ohio

10.3
A Document Engineering Environment for Clinical Guidelines, http://www.guidlihne.gov/
Bridging the Guideline Implementation Gap: A Systematic, Document-Centered Approach to Guideline Implementation, Journal of the American Medical Informatics Association, Volume 11, Number 5, September/October 2004
Proposal for Fulfilling Strategic Objectives of the U.S. Roadmap for National Action on Decision Support through a Service-oriented Architecture Leveraging HL7 Services, Journal of the American Medical Informatics Association, Volume 14, Number 2, March/April 2007
Reasoning Foundations of Medical Diagnosis: Symbolic logic, probability, and value theory aid our understanding of how physicians reason., 3 Jul. 1959, Volume 130, Number 3366, Science

10.4
Contextualization as an Independent Abstraction Mechanism for Conceptual Modeling, Information Systems Journal
Context-aware Process Design: Exploring the Extrinsic Drivers for Process Flexibility, In Latour, Thibaud and Petit, Michael, Eds. Proceedings 18th International Conference on Advanced Information Systems Enginnering. Proceedings of Workshops and Doctoral Consortium., pages pp. 149-158.

10.6
Design of a clinical event monitor, Hripcsak G, Clayton P D, Jenders R A, Cimino J J, Johnson S B., Comput Biomed Res. 1996 June; 29(3):194-221.
A Systematic Review of the Performance Characteristics of Clinical Event Monitor Signals Used to Detect Adverse Drug Events in the Hospital Setting, Steven M. Handler MD, MS1*, Richard L. Altman MD2, Subashan Perera PhD3, Joseph T. Hanlon PharmD, MS4, Stephanie A. Studenski MD, MPH5, James E. Bost MS, PhD6, Melissa I. Saul MS7, and Douglas B. Fridsma MD, PhD7, Journal of the American Medical Informatics Association 2007; 14(4):451-458
Hope C, Overhage J M, Seger A, Gandhi T K, Bates D W, Murray M D, et al. A tiered approach is more cost effective than traditional pharmacist-based review for classifying computer-detected signals as adverse drug events. Journal of Biomedical Informatics. 36(1-2):92-8, 2003 Feb-Apr.

10.7
Service-oriented Architecture in Medical Software: Promises and Perils, J Am Med Inform Assoc. 2007; 14:244-246.

11 Needs Analysis, Ethnographic methods, Contextual/Interaction Design
Medical Informatics and the Science of Cognition., Journal of the American Medical Informatics Association Volume 5 Number 6 November/December 1998
Working minds: A practitioner's guide to cognitive task analysis., MIT Press. Crandall, B., Klein, G., and Hoffman, R. (2006).
A guide to task analysis., Kirwan, B. and Ainsworth, L. (Eds.) (1992). Taylor and Francis.
User and Task Analysis for Interface Design., Hackos, JoAnn T. and Redish, Janice C. (1998). Wiley.
Writing Better Computer User Documentation—From Paper to Online., Brockmann, R. John (1986). Wiley-Interscience.
The Nurnberg Funnel—Designing Minimalist Instruction for Practical Computer Skill., Carroll, John M. (1990). MIT.
Marion Buchenau & Jane Fulton Suri, “Experience Prototyping”, DIS '00, ISBN 1-58113-219-0/00/0008.
Alan Cooper & Robert M. Reimann: About Face 2.0: The Essentials of Interaction Design, Wiley, 2003, ISBN 0-764-52641-3.
Stephanie Houde & Charles Hill, “What Do Prototypes Prototype?” in Handbook of Human-Computer Interaction (2nd ed.), M. Helander, T. Landauer, and P. Prabhu (eds.), Elsevier Science B. V, 1997.
Brenda Laurel & Peter Lunenfeld: Design Research: Methods and Perspectives, MIT Press, 2003, ISBN 0-262-12263-4.
Bill Moggridge, Designing Interactions, MIT Press, 2007, ISBN 0-262-13474-8.
Donald Norman: The Design of Everyday Things, ISBN 0-465-06710-7.
Jef Raskin: The Humane Interface, ACm Press, 2000, ISBN 0-201-37937-6.
Dan Saffer: Designing for Interaction, New Riders, 2006, ISBN 0-321-43206-1.
Beyer, H. & Holtzblatt, K. (1998). Contextual Design: Defining Customer-Centered Systems. San Francisco: Morgan Kaufmann. ISBN: 1-55860-411-1
Weinberg, J. B. and Stephen, M. L. 2002. Participatory design in a human-computer interaction course: teaching ethnography methods to computer scientists. In Proceedings of the 33rd SIGCSE Technical Symposium on Computer Science Education (Cincinnati, Ky., Feb. 27-Mar. 3, 2002). SIGCSE '02. ACM Press, New York, N.Y., pp. 237-241
Holtzblatt, K., Wendell, J. B., & Wood, S. 2005. Rapid Contextual Design: A How-to guide to key techniques for user-centered design. San Francisco: Morgan-Kaufmann.
Akao, Yoji [1994]. “Development History of Quality Function Deployment”, The Customer Driven Approach to Quality Planning and Deployment. Minato, Tokyo 107 Japan: Asian Productivity Organization, 339. ISBN 92-833-1121-3.
Lou Cohen. 1995. Quality Function Deployment. Prentice Hall PTR, ISBN 0201633302.
Spradley, James P. (1979) The Ethnographic Interview. Wadsworth Group/Thomson Learning.
Salvador, Tony; Genevieve Bell; and Ken Anderson (1999) Design Ethnography. Design Management Journal.

12 EDiT
“Maximum likelihood from incomplete data via the EM algorithm”., Journal of the Royal Statistical Society, Series B, 39(1):1-38, 1977
Fast algorithms for mining association rules (1994) by Rakesh Agrawal, Ramakrishnan Srikant, In Proc. of Int. conf. Very Large DataBases (VLDB'94
Cristen, P & T: Febrl—Freely extensible biomedical record linkage (Manual, release 0.3), http://datamining.anu.edu.au/linkage.html
“Record Linkage”, American Journal of Public Health 36 (12): pp. 1412-1416.
Mirth Project Open Source HL7 Integration Engine, http://www.mirthprojectorg/
HL7 Integration Engine, http://www.neotool.com/blog/2007/05/14/why-use-an-hl7-engine/

13 Knowledge Management System
Greenes, R A. Clinical Decision Support: The Road Ahead New York. Elsevier. 2007.
AMIA: A Roadmap for National Action Plan on Clinical Decision Support. Jun. 13, 2006., Available from: http://www.amia.org/inside/initiatives/cds/cdsroadmap.pdf
Information Theory, Inference, and Learning Algorithms, http://www.inference.phy.cam.ac.uk/mackay/itila
Akscyn, Robert M., Donald L. McCracken and Elise A. Yoder (1988). “KMS: A distributed hypermedia system for managing knowledge in organizations”., Communications of the ACM 31 (7): 820-835.
Knowledge Management Systems: Information And Communication Technologies for Knowledge Management., Maier, R (2007): 3rd edition, Berlin: Springer.
Schadow G. HL7 Structured Product Labeling—electronic prescribing information for provider order entry decision support., AMIA Annu Symp Proc. 2005:1108
Food and Drug Administration. Requirements for submission of labeling for human prescription drugs and biologics in electronic format [21 CFR Parts 314 and 601]., Federal Register. 2003 68(238):69009-20.December 11;
Office of the Secretary HHS. Consolidated Health Informatics (CHI) Initiative; Health Care and Vocabulary Standards for Use in Federal Health Information Technology Systems., Federal Register. December 2005; 70(246): 76287 8
Aronson A R. Effective mapping of biomedical text to the UMLS Metathesaurus: the MetaMap program., Proc AMIA Symp. 2001:17 21. See also http://mmtx.nlm.nih.gov/
Overhage J M, Tierney W M, Zhou X H, McDonald C J. A randomized trial of “corollary orders” to prevent errors of omission., J Am Med Inform Assoc.; 4 (5):364 75
Dexter P R, Perkins S M, Maharry K S, Jones K, McDonald C J. Inpatient computer based standing orders vs physician reminders to increase influenza and pneumococcal vaccination rates., JAMA 2004; 292:19; 2366 2371.
McDonald C J, Hui S L, Smith D M, Tierney W M, Cohen S J, Weinberger M. Reminders to physicians from an introspective computer medical record. A two year randomized trial. Ann Intern Med. 1984 January; 100(1):130 38.
The Interface between Information, Terminology, and Inference Models, Medinfo. 2001; 10(Pt 1):246-50.
Algorithms for association rule mining—a general survey and comparison, ACM SIGKDD Explorations Newsletter; Volume 2, Issue 1; Pages: 58-64

Vocabulary Services
Desiderata for Controlled Medical Vocabularies in the Twenty-First Century Methods, Inf Med. 1998 November; 37(4-5):394-403.
From Data to Knowledge through Concept-oriented Terminologies, J Am Med Inform Assoc. 2000 May-June; 7(3):288-97
A Semantic Lexicon for Medical Language Processing, Journal of the American Medical Informatics Association 6:205-218 (1999)
Using contextual and lexical features to restructure and validate the classification of biomedical concepts, BMC Bioinformatics 2007, 8:264
WordNet, http://wordnet.princeton.edu

Finally, it should be understood that references to C.R.E. (Context Reality Engine) in the provisional application now refer to the CC engine. The currently described vocabulary services were referred to as Vocabulary Engine in the provisional application. Also, the currently described Gaming Environment was referred to as the Serious Game Environment in the provisional application.

hile this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A machine readable medium having machine-executable instructions for managing workflow of an enterprise, the machine-executable instructions including:
means for applying context to information relating to workflow activities of the enterprise;
means for establishing meaning of the information;
means for linking the information;
means for deriving associations relating to the information;
means for generating a model of the workflow based on the information; and
means for providing a simulation of the workflow based on the model.

2. A method for managing a workflow of activities of enterprise, including the steps of:
using a computing device to define a current state of enterprise activities by gathering information from at least one tracking system coupled to the computing device;
using the computing device to determine how the workflow appears to be done;
using the computing device to determine, based on the preceding steps, how the workflow is really being done by defining a model from the current state of the enterprise activities and the determination of how the workflow appears to be done; and
adjusting variables affecting the workflow to determine, using the computing device, how the workflow should be done at the enterprise;
wherein the step of adjusting variables includes the step of refining the model by providing feedback to the model definition as a result of simulation including interaction with a game environment that provides one of virtual and mixed reality game situations to predict outcomes based on the interactions, the predicted outcomes being provided as the feedback to the model definition.

3. The machine readable medium of claim 1, wherein the means for establishing meaning of the information includes instructions which when executed provide at least one of a vocabulary service and a terminology map.

4. The machine readable medium of claim 3, wherein the terminology map provides a relationship between local terminologies and established standards.

5. The machine readable medium of claim 1, wherein the means for applying context includes instructions for associating the context of the information with a contextual property from the group consisting of role, environment, and activity.

6. The machine readable medium of claim 1, wherein the means for linking includes instructions which when executed employ a probabilistic linkage technology.

7. The machine readable medium of claim 1, wherein the means for deriving associations includes instructions which when executed perform rule mining to identify characteristics of the workflow activities.

8. The method of claim 2, wherein the tracking system is one of an optical tracking system, an IR tracking system, an RF tracking system, and a position sensing system.

9. The method of claim 2, wherein the defining step includes the step of defining parameters for at least one process of the workflow, the parameters being derived from Apriori knowledge sources that are independent of the enterprise.

10. The method of claim 9, wherein the Apriori knowledge sources include at least one of accepted clinical pathways, accepted clinical guidelines, and known workflows.

11. The method of claim 2, wherein the step of determining how the workflow appears to be done includes the step of using a workflow analysis technique from the group consisting of a walk through with time-motion studies, task analysis, ethnographic inquiries, process mapping, and general data mining, visualization and business intelligence tools.

12. The method of claim 11, wherein the step of using a workflow analysis technique is repeated in an iterative manner.

13. The method of claim 2, wherein the step of adjusting variables includes the step of using petri net mathematics to refine the model.

14. The method of claim 2, wherein the step of adjusting variables includes the step of applying a stochastic modeling technique to the model to estimate a probability distribution of an outcome of the model resulting from random changes in the variables over time.

15. The method of claim 2, further including the step of providing clinical decision support to assets relating to the enterprise.

16. The method of claim 15, wherein the assets include individuals engaged in the activities.

17. The method of claim 15, wherein the assets include information systems external to the enterprise.

18. The method of claim 15, wherein the step of providing clinical decision support includes the step of generating a message for communication from one location in the enterprise to another location in the enterprise.

19. The method of claim 15, wherein the step of providing clinical decision support includes the step of sending a notification to a person associated with the enterprise in a manner that conforms to predetermined preferences of the person.

20. The method of claim 15, wherein the step of providing clinical decision support includes the step of performing medication reconciliation.

21. The method of claim 15, wherein the step of providing clinical decision support includes the step of screening for drug contraindication.

22. The method of claim 15, wherein the step of providing clinical decision support includes the step of screening for drug duplication or therapeutic failure.

23. The method of claim 15, wherein the step of providing clinical decision support includes the step of checking the dose of a drug.

24. The method of claim 15, wherein the step of providing clinical decision support includes the step of managing corollary orders.

25. The method of claim 15, wherein the step of providing clinical decision support includes the step of extracting information from a knowledge base.
